(19) United States
(12) Patent Application Publication
(10) Pub. No.: US 2015/0299086 A1
(43) Pub. Date: Oct. 22, 2015

(54) METHOD FOR THE PRODUCTION OF POLYETHYLENE TEREPHTHALATE WITH A LOW CARBON FOOTPRINT

(71) Applicants: ETH ZURICH, Zurich (CH); CEDAR ADVANCED TECHNOLOGY GROUP LTD., Zug (CH)

(72) Inventors: Said FARHA, Thalwil (CH); Kamal FARHA, Pleasantville, NY (US); Philip FARHA, Ancaster (CA)

(21) Appl. No.: 14/646,742

(22) PCT Filed: Nov. 21, 2013
(86) PCT No.: PCT/EP2013/003511

§ 371 Date: May 22, 2015

(30) Foreign Application Priority Data
Nov. 23, 2012 (EP) 12007917.3

(51) Int. Cl. C07C 51/265; C07C 7/08; B01D 3/40; C07C 29/151; C01B 31/08; C01B 31/20; C08G 63/183; C07C 45/50

(52) U.S. Cl. CPC C07C 51/265; C08G 63/183; C07C 7/08; C07C 45/505; C07C 29/1518; C01B 31/081; C01B 31/20; B01D 3/40

(57) ABSTRACT

The present invention relates to a method for producing terephthalic acid from lignocellulosic biomass including the steps of pyrolysis of lignocellulosic biomass in the presence of a catalyst obtaining biochar, crude bio-oil and gases; collecting an aromatic fraction from the crude bio-oil and/or from the gases, extracting toluene from the aromatic fraction by distillation; converting the toluene to a p-tolualdehyde by catalyzed carbonylation; oxidation of the p-tolualdehyde in a liquid phase oxidation to tereph thalic acid.

The present invention relates to the production of terephthalic acid from lignocellulosic biomass, and in particular to the production of polyethylene terephthalate (PET). Terephthalic acid, polyethylene terephthalate as well as byproducts obtained by the process according to the present invention have a low carbon footprint.

Terephthalic acid is widely used in the manufacture of polyesters, commonly by reaction with ethylene glycol, higher alkylene glycols or combinations thereof, for conversion to bottles, fibers, films, containers and other packaging materials, and molded articles.

In commercial practice, terephthalic acid is commonly made by liquid phase oxidation in an aqueous acetic acid solvent of p-xylene, with air or another source of oxygen, which is normally gaseous, in the presence of a bromine-promoted catalyst comprising cobalt and manganese ions. The oxidation is exothermic and yields the terephthalic acid together with high- and low-molecular weight byproducts.

An alternative preparation for terephthalic acid is a two-step process developed by Mitsubishi Gases in the 1970s. The first step is the production of p-tolualdehyde by HF/BF₃ catalyzed carbonylation of toluene. The second step is a liquid phase oxidation of tolualdehyde to terephthalic acid. This process is described for example in U.S. Pat. No. 4,245,078. However, this process could never replace the commercially used process for the production of terephthalic acid by p-xylene.

U.S. Pat. No. 4,554,383 discloses a process for the selective carbonylation of toluene to p-tolualdehyde which comprises mixing an N-alkylpyridinium halide and an anhydrous aluminium halide melt catalyst with toluene at room temperature of higher and at superatmospheric pressure until the tolualdehyde is formed and separated.

U.S. Pat. No. 5,679,847 discloses a process for producing terephthalic acid by oxidizing the mixture of p-xylene and p-tolualdehyde as the starting raw material by the use of a molecular oxygen-containing gas by using a lower aliphatic monocarboxylic acid as a solvent in the presence of a heavy metal compound and a bromine compound. Terephthalic acid obtained by this process has a high whiteness.

U.S. Pat. No. 3,956,694 discloses a process for purifying p-tolualdehyde from a mixture of tolualdehyde by cooling the mixture to a crystallization temperature of −6° C. to −60° C., thereby crystallizing p-tolualdehyde, and separating the resulting p-tolualdehyde crystals from mother liquor.

Further Henkel developed two processes to obtain terephthalic acid. The Henkel I process is the manufacture of dipotassium phthalate from phthalic anhydride, which is then rearranged in an isomerization step to dipotassium terephthalate at 430-440° C. and 5-20 bar CO₂ in the presence of a Zn—Cd catalyst. The potassium recycle is conducted with a potassium exchange between K terephthalate and phthalic anhydride. In the Henkel II process, potassium benzoate is disproportionated at 430-440° C. in the presence of CO₂ (50 bar) and Cd or Zn benzoate to dipotassium terephthalate and benzene with 95% selectivity. The starting compound for the Henkel I process, phthalic anhydride, can be obtained by catalytic oxidation of o-xylene and naphthalene. The catalyst that is used for the oxidation of xylene is a modified vanadium pentoxide (V₂O₅) . The phthalic anhydride can be separated from byproducts such as o-xylene in water, or maleic anhydride, a series of condensers.

p-xylene is produced by catalytic reforming of petroleum naphtha as part of the BTX aromatics (benzene, toluene and the xylene isomers) extracted from the catalytic reformate in big sized and extremely investment-intensive production plants. The p-xylene is then separated out in a series of distillation, adsorption or crystallization and reaction processes from the m-xylene, o-xylene and ethyl benzene. Its melting point is the highest among this series of isomers, but simple crystallization does not allow easy purification due to the formation of eutectic mixtures.

There is still a lot of research carried out to improve the process for preparing terephthalic acid from p-xylene as starting compound. For example WO 2012/012047 discloses a process for producing terephthalic acid from p-xylene. The process comprises forming a mixture comprising p-xylene, a solvent, a bromine source and a catalyst; and oxidizing the p-xylene by contacting the mixture with an oxidizing agent at oxidizing conditions to produce a solid oxidation product comprising terephthalic acid, p-toluic acid and 4-carboxybenzaldehyde.

Terephthalic acid derived from petroleum has a high carbon footprint, which is particularly unwanted due to the very large amounts produced and used today. The production of p-xylene as starting material is extremely energy consuming, thereby resulting in said high carbon footprint. In addition, there is a high dependence on the petroleum industry, and due to the low number of competitors in this industry there are few possibilities to reduce the prices of the starting compounds.

WO 2009/064515 discloses terephthalic acid prepared by reacting a 2,5-furandicarboxylate with ethylene in the presence of a solvent to produce a bicyclic ether; then dehydrating the bicyclic ether. 2,5-furandicarboxylate is derived from biomass, whereby enzymatic or microbial degradation occurs from biomass carbohydrates to produce fructose, sucrose and mixtures thereof, the sugars are then converted to 5-hydroxymethylfurfural, and the 5-hydroxymethylfurfural is readily oxidized to 2,5-furandicarboxylate. Although this terephthalic acid may be produced with a low carbon footprint, the process is not efficient enough, it is time consuming and expensive and is therefore not suitable for producing large amounts of terephthalic acid.

Biomass can be converted into renewable energy sources and chemicals via thermal, biological, chemical, or physical processes. Three main types of thermal conversions of biomass are known, that is combustion, gasification, and pyrolysis. Pyrolysis can be defined as an anaerobic thermochemical decomposition of biomass.

U.S. Pat. No. 8,137,628 discloses a system and method for the conversion of biomass to renewable fuels. By renewable fuel is meant any combustible fuel that is derived from biomass and that is useful for transportation or other purposes, including such fuels as gasoline, diesel, jet fuel, or other useful blends, such as blends of benzene, toluene and xylene (BTX).

WO 2012/022949 discloses a process for the pyrolysis of lignin comprising the steps of feeding a paste comprising lignin into a reaction chamber and performing pyrolysis on the paste in the reaction chamber. The process can be used for the production of phenolic compounds. Small amounts of aromatic hydrocarbons such as benzene, toluene and indene are produced as by-product of said process, due to the hydrogenation of the phenolic compounds.

The problem of the present invention is to provide a method for producing terephthalic acid with a low carbon footprint, that is, green terephthalic acid in small sized production sites.

The problem is solved by the method according to claim 1. Further preferred embodiments are subject to dependent claims.

Surprisingly, it was found that terephthalic acid can be produced from lignocellulosic biomass as feedstock instead of using large amounts of petroleum. Said method includes the steps of
a. pyrolysis of lignocellulosic biomass in the presence of a catalyst obtaining biochar, crude bio-oil and gases;
b. collecting an aromatic fraction from the crude bio-oil and/or from the gases;
c. extracting renewable toluene from the aromatic fraction by distillation;
d. converting the toluene to a p-tolualdehyde by catalyzed carbonylation;
e. oxidation of the p-tolualdehyde in a liquid phase oxidation to terephthalic acid.

The method according to the present invention provides the possibility to obtain terephthalic acid from lignocellulosic biomass, and in particular from food waste. Lignocellulosic biomass is renewable, meaning their sources can be regrown. Further, millions of tons of food waste are available. The method according to the present invention further offers environmental benefits such as lower carbon and lower sulfur emissions compared with conventional produced petroleum-based terephthalic acid. Since the process according to the present invention is straightforward and can be carried out in existing infrastructure, a large-scale production is economical and will enable a rapid market acceptance. In contrast to the state of the art no large refineries are necessary and more importantly, the production of terephthalic acid is independent from the petroleum industry. In addition, the method according to the present invention results in a pure product with high yield. The so obtained terephthalic acid can be converted directly to polyethylene terephthalate or to another polyester without additional purification. Polyethylene terephthalate is obtained by reacting the terephthalic acid obtained by the method according to the present invention with ethylene glycol under standard conditions. However, it is also possible to purify the polyethylene terephthalate further.

The first step in the process (step a) according to the present invention is a pyrolysis reaction of lignocellulosic biomass. Said reaction results in three main components, namely biochar, crude bio-oil, including the vapor of said bio-oil, and gases. The crude bio-oil comprises an aromatic fraction from which toluene may be separated by distillation. Said three main components consist of several substances, each of which may be used as chemical feedstock in the future dependent on the customer's needs. All substances are produced with a low carbon footprint. Interestingly, the process according to the present invention does not only allow an effective production of terephthalic acid but also permits, if desired, the use of all byproducts, such as carbon monoxide, carbon dioxide, methane, hydrogen, biochar and residues of crude bio-oil, occurring during pyrolysis as chemical feedstock or as energy source.

Within the context of the present invention the term crude bio-oil stands for the liquid component obtained after pyrolysis. Crude bio-oils are typically dark brown free flowing liquids, similar in their appearance to petroleum derived crude oils. Their elemental composition is usually approximate to that of the starting biomass. The yields and properties of the liquid bio-oil depend on the feedstock, the process type and operating conditions, and the product collection efficiency. Especially preferred is a feedstock having a high lignin content, since this results in a high amount of the aromatic fraction. The expression high lignin content stands for example for a feedstock lignin content of more 25% and more preferably more than 35% based on the total weight of cellulose, hemicellulose and lignin. The crude bio-oil obtained by the process according to the present invention has typically a viscosity of 40 to 100 cp, containing up to 0.5% by weight biochar. A low pH of roughly 2.5 is common due to the presence of organic acids. Said organic acids can facilitate acid catalyzed aldol reaction between aldehydes and ketones. Thus, crude bio-oil tends to change composition over time and has also an increasing viscosity over time. Therefore, the collection efficiency is very important.

The liquid bio-oil comprises about five families of compounds. The first family consists of very volatile organic compounds, mainly hydroxylacetaldehyde, formic acid, and methanol. The second family consists of water and other volatile compounds such as acetic acid, acetol and propionic acid. The third, and for this invention the most important family is made up of compounds with moderate volatility (boiling points between 100 and 250° C.) representing preferably up to 45 mass % of the crude bio-oil. These compounds are furans and mono aromatic compounds, such as BTX, generated from depolymerization reactions. The fourth family consists of compounds with boiling points in the range of 200 to 300° C. and accounts for up to 35% by weight of the crude bio-oil. Five major groups of compounds are found in this family: polyaromatics (10 to 28 carbon atoms), aliphatic hydrocarbons (13 to 32 carbon atoms), fatty acids (14 to carbon atoms), sterols and sugars (mostly levoglucosan). The last family can account for up to 35% by weight of the crude bio-oil, and is made up non-volatile components, oligomeric sugars and phenolics with molecular masses between 600 and 10,000 g/mol.

In the method according to the present invention the feedstock is lignocellulosic biomass, that is, the feedstock is not in competition with food. Biomass is virtually the only “carbon neutral” or “carbon negative” renewable resource. Additionally lignocellulose is the most inexpensive and most abundant bio-resource available.

Lignocellulosic biomass comprises cellulose, hemicellulose, lignin and minerals, also known as ash. The complex mixtures of compounds found in the bio-oil are derived from the cleavage of cellulose, hemicellulose, and lignin chemical bonds, occurring in well-defined temperature ranges. These reactions are called primary thermal decomposition reactions.

Preferably, the lignocellulosic biomass used in the pyrolysis step according to the present invention is food waste, wood residues or municipal paper waste.

The expression food waste includes losses during agricultural productions that is due to mechanical damage and/or spillage during harvest operation (e.g. threshing), crops sorted out post harvest, losses during postharvest handling and storage, that is due to spillage and degradation during handling, storage and transportation between farm and distribution, losses during processing, that is due to spillage and degradation during industrial or domestic processing, e.g. canning and bread baking (losses may occur when crops are sorted out, if not suitable to process or during washing, shelling, slicing and boiling or during process interruptions and accidental spillage), losses during distribution in the market system, at e.g. wholesale markets, supermarkets, retailers and wet markets, as well as losses during consumption at the household level.

Preferably, the food waste is the food waste selected from the group of non-edible food waste and waste occurring during agricultural production, waste occurring during postharvest handling and storage, waste occurring during processing, waste occurring during distribution and waste occurring during consumption or mixtures thereof. In a most preferred embodiment, the food waste is selected from waste occurring during agricultural production, and waste occurring during processing, since these food wastes are located in large amounts on the same place, which allows fast collection of big amounts of the food waste.

Especially preferred is the food waste occurring during processing in food industry.

Preferably, the lignocellulosic biomass is food waste selected from the group of coconut shells, pecan shells, almond shells, rice straw, wheat straw, rice hulls, corn stover, corn straw, coffee draff, cocoa shells, potato peels and corn crops or mixtures thereof.

Preferably, the pyrolysis unit contains only one specific kind of biomass, most preferably one specific kind of food waste, since the content of the crude bio-oil and of the biochar is strongly dependent on the feedstock. Therefore, dependent on whether for example mainly toluene has to be extracted or activated carbon has to be produced from biochar the best feedstock for this application can be chosen.

In contrast to the commercially used process for the production of terephthalic acid, the process according to the present invention uses toluene and not p-xylene as synthesis starting compound for the production of terephthalic acid. This allows the disentanglement of the production of terephthalic acid from using the complex refinery driven processes associated with xylene extraction and large plants. When carrying out the pyrolysis of the lignocellulosic biomass, the obtained aromatics in the crude bio-oil (that is the fraction comprising compounds with moderate volatility having boiling points between 100 and 250° C.) comprise at least 25%, preferably at least 30%, most preferably at least 35% toluene and 5% to 15% xylene. For example, it can comprise 37.9% toluene and 12% xylene. Said 12% xylene comprise o-, m- and p-xylene, that is, said compounds would have to be separated by distillation again, which is difficult, since they have similar boiling points. Therefore, toluene as starting compound is much more preferred.

The pyrolysis of the lignocellulosic biomass, the collection of the aromatic fraction and the extraction of the toluene can be carried out according to several methods known in the art. For example it can be carried out according to the method described in U.S. Pat. No. 8,137,628, wherein the renewable fuel obtained after carrying out the pyrolysis is distilled to obtain toluene. The content of this document is herewith incorporated in its entirety, in particular column 4, line 58 to column 8, line 14.

Alternatively, the pyrolysis can be carried out in a fluidized bed reactor, in a fixed bed reactor or a semi-batch reactor, whereas a fluidized bed reactor is preferred. However, preferably the pyrolysis unit has a small size, for example 2 to 20 tons per day, allowing a fast collection and avoiding unwanted side reactions.

Before carrying out the pyrolysis, the lignocellulosic biomass can optionally be dried. In addition, the size of the biomass can optionally be reduced by chipping, shredding, grinding and/or milling. Alternatively, it is also possible to carry out a chemical pretreatment by addition of acids, bases, organic solvents and/or ionic liquids. It is also possible to use the lignocellulosic biomass without pretreatment.

The pyrolysis is carried out in the presence of a catalyst (also called catalytic fast pyrolysis), in order to obtain a more desirable hydrocarbon content. Preferably the pyrolysis is carried out at a temperature from 300° C. to 500° C., most preferably at 325° C. to 400° C. and preferably at a pressure of 1 to 5 bar, most preferably at 2 to 3 bar. In order to avoid side reactions, the pyrolysis is preferably carried out at low temperatures from 300° C. to 500° C., while collecting the aromatic fraction continuously or in short time intervals of about 30 to 90 minutes, most preferably 30 to 45 minutes.

Preferably the catalyst is an aromatization catalyst. Examples of aromatization catalysts are silicate-1, alumina-silica, AlPO₄ molecular sieves, MFI type zeolites such as H—ZSM-5 (zeolite socony mobil), H—Y, and metal modified MFI (mordenite framework inverted) type zeolites, where the metal is selected from the group consisting of: Group VIB metals, Group VIIB metals, Group VIII metals,

Group IB metals, Group IIB metals, Ga, In, and all combinations thereof. Zeolite, namely the H—ZSM-5 catalyst, has been shown to generate very high organic fractions, containing 87% by weight of hydrocarbons with aromatic yields of approximately 16% by weight, toluene and xylenes being the dominant species. Additionally, the inherently high acidic character of the H—ZSM-5 zeolite results in strong dehydration tendency, making it an effective oxygen removal catalyst. Further, by minimizing coke formation aromatic yields can be improved.

In one embodiment of the present invention the pyrolysis of the lignocellulosic biomass is carried out in several pyrolysis units. Every pyrolysis unit is operated at a different temperature, whereby the temperature of each subsequent pyrolysis unit increases. However, it is also possible that all pyrolysis units operate at the same temperature. Better results have been obtained with different temperatures. Preferably the temperature difference between two adjacent pyrolysis units is about 10° C. to 100° C., preferably 40° C. to 60° C., most preferably 50° C. or 60° C. The starting temperature in the first unit is determined by the initial composition of lignocellulosic biomass and is typically between 250° C. and 400° C., preferably between 300° C. and 350° C. The end temperature is typically between 500° C. and 600° C. The preferred number of pyrolysis units also depends on the initial composition of the biomass, and is typically between 5 and 20, preferably between 5 and 10, most preferably 5 or 6. Preferably every pyrolysis unit comprises a catalyst, preferably an aromatization catalyst, whereby the catalyst may be the same, or at least some of the pyrolysis units have a different aromatization catalyst.

The output of each pyrolysis unit comprises usually also volatile gases (vapor of the bio-oil). In order to increase the output of the pyrolysis process the aromatic fraction of the gases is also separated and collected. The volatile gases are programmed to pass through subsequent catalytic columns, which comprise as well an aromatization catalyst to convert the volatile gases into an aromatic fraction comprising BTX.

The aromatic fractions are collected from the crude bio-oil and from the gases. Subsequently the toluene is separated from the aromatic fraction by distillation. The remainder of the crude bio-oil may be used as “green” energy source for other chemical processes, or after further purification as feedstock for chemical reactions.

After distillation the toluene is converted to tolualdehyde by catalyzed carbonylation.

In a typical reaction sequence, toluene, HF and BF₃ are combined at low temperature, preferably at 0° C. to form a complex, into which about 99% pure carbon monoxide is charged under pressure, preferably at 25 to 30 bar. As mentioned below in detail, in a preferred embodiment, the carbon monoxide is obtained during the pyrolysis as well. Upon addition of the carbon monoxide, p-tolualdehyde is obtained in more than 90%, preferably more than 95% and most preferably more than 96% selectivity at toluene.

The process is a variation of the Gattermann-Koch reaction. The p-tolualdehyde complex is thermally decomposed in a distillation column. The overhead from the distillation column is liquid HF which is recycled.

Alternatively the p-tolualdehyde can be prepared by mixing an N-alkylpyridinium halide and an anhydrous aluminium halide melt catalyst with toluene and carbon monoxide at a temperature of 0 to 200° C. and at a pressure of 1 to 300 bar. Preferred catalysts for this process step are disclosed in U.S. Pat. No. 4,554,383. This process is a good alternative, since it avoids the use of liquid HF.

Afterwards the p-tolualdehyde is converted to terephthalic acid by liquid phase oxidation. The liquid phase oxidation reaction is carried out batchwise, semicontinuously or continuously.

The reaction temperature in liquid phase oxidation is in the range of 120° C. to 240° C. In order to make sure that the reaction may be carried out in a liquid phase, the reaction system usually needs to be pressurized so as to maintain the starting raw material as well as the solvent in a liquid phase. Hence a reaction pressure in the range of 1 to 50 bar is usually used. Preferably the reaction is carried out in the presence of a catalyst.

The solvent used for the oxidation reaction is preferably a lower aliphatic monocarboxylic acid such as acetic acid, propionic acid, butyric acid or valeric acid. Acetic acid is particularly preferred as the solvent.

The product solution obtained by the liquid phase oxidation is a slurry containing crystalline terephthalic acid. The slurry may be filtered and the thus obtained crystals may be washed with acetic acid and/or water and then dried to obtain terephthalic acid.

The terephthalic acid can be produced in a yield of 95% by mol or more. A purity of 99% makes the terephthalic acid according to the present invention into a suitable starting compound for the production of polyethylene terephthalate.

In another aspect of the present invention not only the toluene can be used for the production of the terephthalic acid, but also the xylene part of the aromatic fraction. The xylene part is extracted from the aromatic fraction by distillation. The xylene fraction is then oxidized under standard conditions to p-tolualdehyde by carrying out a catalyzed carbonylation. The reaction mixture comprises not only p-tolualdehyde but also remaining o-, m- and p-xylene. In a subsequent step o-, and m-xylene are separated by freezing the mixture at a temperature of less −5° C., preferably less than −6° C., thereby crystallizing p-tolualdehyde and p-xylene. This mixture of p-tolualdehyde and p-xylene can be converted to terephthalic acid by liquid phase oxidation following the disclosure of U.S. Pat. No. 5,679,847. Preferably the reaction is carried out at a temperature of 120° C. to 240° C. in the presence of a molecular oxygen-containing gas, a solvent, said solvent being a lower aliphatic monocarboxylic acid and a catalyst.

In another aspect of the present invention the p-tolualdehyde obtained by catalyzed carbonylation of the toluene and the mixture of p-tolualdehyde and p-xylene obtained as described above are mixed together before converting them to terephthalic acid. Therefore, the yield of terephthalic acid derived from the pyrolysis process can be increased by combining these two aspects of the invention.

In another aspect of the present invention m-xylene, which can either be obtained by separation of the xylene fraction, or by separation from the above mother liquid, can be converted to isophthalic acid according to the methods known to a person skilled in the art. Isophthalic acid is used in PET manufacturing as co-polymer, that is also the co-polymer is fully renewable based, having a low carbon footprint.

Within the context of the present invention the expression biochar refers to the remaining solid component after pyrolysis. Biochar is a porous organic material with a high carbon content, produced via the pyrolysis of biomass. The biomass feedstock and the pyrolysis operating conditions (i.e. heating rate, residence time, pressure, flow rate of the inert gas, reactor type and shape) are the main factors determining chemical composition as well as the physical properties (particle size, pore size, pore volume and surface area of the biochar). A preferred application of biochar is its use as a precursor of activated carbon.

In a preferred embodiment of the present invention the activated carbon is produced by either physical or chemical activation at elevated temperature.

Chemical activation is achieved by first impregnating the lignocellulosic biomass before carrying out the pyrolysis with an activating agent such as H₂SO₄, H₃PO₄, ZnCl₂ or alkali metal hydroxides such as KOH, whereas H₃PO₄ is especially preferred. After impregnation of the lignocellulosic biomass with an activating agent, the pyrolysis reaction is carried out as described before. The activated product is washed and dried, the chemicals are recovered. The activity can be controlled by altering the proportion of the lignocellulosic biomass to activating agent (1:0.5 to 1:4) or by controlling temperature and residence time.

Alternatively the biochar obtained after the pyrolysis of the lignocellulosic biomass is impregnated by an activating agent selected from the group consisting of H₂SO₄, H₃PO₄, ZnCl₂ or alkali metal hydroxides such as KOH or mixtures thereof, most preferably by KOH. In a second step the impregnated biochar is further carbonized at elevated temperature of 700° C. to 900° C., preferably 800° C. in an inert atmosphere.

The activated carbon obtained by both of the above processes has an internal surface area of 1400 to 1600 m²/g and a highly microporous structure of 60 to 90%, preferably of about 80%.

In another embodiment, the activated carbon is obtained by an activation step after pyrolysis. The biochar is activated in oxidizing gases such as carbon dioxide, steam or air at temperatures of 800° C. to 1000° C., preferably 900° C. producing the final activated carbon. The activation step can last from half an hour to 10 hours, preferably 3 to 5 hours. Preferably, as oxidizing gas carbon dioxide obtained during pyrolysis is used.

The degree of activation is directly dependent on the biochar and its initial surface area. Best results could be obtained with biochar obtained from coconut shells due to the significant number of pores per mm².

It is known to those skilled in the art that activated carbon is one of the most effective adsorbents, due in parts to well-developed porous structure, large surface area and good mechanical properties. Since lignocellulosic biomass often occurs in developing countries having large quantities of food waste, it is possible to produce activated carbon for the purification of water locally at low costs.

Within the context of the present invention the expression gases stands for gases obtained during the pyrolysis such as carbon monoxide, carbon dioxide, hydrogen and methane, as well as aromatic vapors and olefins as well as for the vapor of the bio-oil. All of these gases can be isolated and be used as chemical feedstock or energy source.

In a further embodiment of the present invention the carbon monoxide (CO) is extracted from the gases during the pyrolysis and used for example for the carbonylation of the toluene to p-tolualdehyde. This allows a just in time production of the necessary starting compounds.

Before using the carbon monoxide out of the process the carbon monoxide has to be purified. The initial step is usually the removal of minor impurities. Particulates are typically removed in cyclones or by scrubbing and acid gases are typically removed by absorption. After initial purification, the gas stream is sent to a carbon monoxide recovery section for final purification, where byproducts such as carbon dioxide are also recovered.

The final purification can be carried out by a method known to the person skilled in the art such as cryogenic purification, pressure swing adsorption, membrane separation or salt solution absorption.

In a preferred embodiment the purification is carried out by cryogenic separation (cold box). This can be achieved by partial condensation techniques or by liquid methane wash. Both operate at cryogenic temperatures and elevated pressures and rely on the expansion energy in the feed gas to produce most or all of the refrigeration energy required. The so obtained carbon monoxide has a purity of greater than 99%, which means that the carbon monoxide can be used in the carbonylation reaction without further purification.

In another embodiment the purification is carried out by the cosorb process of the firm Tenneco Chemicals (see Chem. Engineering, 4 Aug., 1975, p. 52). It is based on a principle that a solution comprising organometallic complexes which is capable of absorbing one mol of carbon monoxide per mol copper(I). The preferred embodiment employs a cuprous tetrachloroaluminate toluene complex (CuAlCl₄*C₆H₅CH₃) in a toluene solvent. Carbon monoxide is recovered in a regenerator by heating the rich solution to approximately 80° C., at atmospheric pressure. Carbon monoxide can be obtained with a purity up to 99.7%. The carbon monoxide purity is very high because the physically absorbed gases are removed prior to the stripping column. Also, the absorbed carbon monoxide cannot be oxidized. Alternatively, the purification can also be carried out by zeolite, such as by zeolite types a, B, X and LSX (Low Silica X) and Y, in particular zeolites belonging to the group of faujasites (type Y, X, ALSX) or to the group of A-type zeolites (LTA). Zeolite types 4A, 5A, 13X, chabazite (e.g. SSZ-13) and NAKLSX are particularly preferred.

Preferably, the carbon monoxide is purified for on-site use, meaning that the carbonylation of the p-tolualdehyde is carried out in the same production site.

Alternatively or in addition, the so obtained carbon monoxide as well as hydrogen separated from the gas stream may be used as starting compounds for the production of ethylene glycol, which is used for the production of polyethylene terephthalate from terephthalic acid. The reaction can be carried out through an acid-catalyzed carbonylation of formaldehyde, using a strong acid catalyst (Nafio® solid perfluorosulfonic acid resin). The glycolic acid product of this carbonylation is esterified and then hydrogenated to yield ethylene glycol (Dan. E. Hendriksen, Intermediates to ethylene glycol Prepr. Pap.—Am. Chem. Soc., Div. Fuel Chem., 28(2), 176-90 (English) 1983).

Therefore, with the process according to the present invention it is possible to obtain toluene and carbon monoxide as starting compounds for the production of terephthalic acid and in addition carbon monoxide and hydrogen as starting compounds for the preparation of ethylene glycol. Thus for esterification reaction between terephthalic acid and ethylene glycol with water as a byproduct resulting in polyethylene terephthalate, starting compounds with a low carbon footprint can be provided.

In another embodiment of the present invention the carbon dioxide (CO₂) is extracted from the gases during the pyrolysis. The separation of carbon dioxide from a gaseous stream is known in the art. This can be done by physical or chemical adsorption such as cryogenic separation and membrane separation. Preferably, the carbon dioxide separation is carried out by a mesoporous carbon dioxide basked as disclosed in Prep-. Pap. Am. Chem. Soc., Div. Fuel Chem. 2004, 49 (1) 259, wherein carbon dioxide-philic materials, such as sterically branched polymer polyethyleneimine (PEI) are loaded into mesoporous molecular sieve MCM-41. The so obtained carbon dioxide may be used as chemical feedstock in future. A preferred application is in beverage industry for the production of carbonated beverages. Carbon dioxide obtained by the pyrolysis reaction according to the present invention and subsequent isolation of the gas has a low carbon footprint as well.

Another aspect of the present invention refers to a production unit comprising one or more pyrolysis units. Each pyrolysis unit comprises a catalyst, preferably an aromatic catalyst. Preferably said production unit comprises 1 to 12, most preferably 6 to 8 pyrolysis units, in which each pyrolysis unit is small, that is, having a capacity of 10 to 60 kg, preferably 20 to 30 kg biomass per hour. Such small pyrolysis units allow the production of specific end products without having significant overheads of big units.

In one embodiment the pyrolysis step and collection step may be carried out in a modular production unit. Therefore, the modular production unit comprises one or more pyrolysis units as described above. In addition it may additionally comprise a cyclone for the collection of biochar, and preferably also a kiln to obtain activated carbon. If the modular production unit comprises beside the at least one pyrolysis unit a cyclone and/or a kiln, they are normally not directly connect to each other, and may be used independently from each other.

Because a pyrolysis unit having a small size is preferred, the modular unit can be installed on the trailer of a flat-bed 18-wheel truck, making it mobile and thus transportable to different places with lignocellulosic biomass. This feature makes it ideal to access the food waste that is often left stranded in agricultural regions, far away from industrial facilities. A modular production unit is especially important in developing countries, which have a lot of lignocellulosic biomass, but said biomass is distributed in small amounts over the whole country. Examples are small cocoa or coffee plantations, which produce a lot of biomass which could be used for the production of terephthalic acid and consequently for the production of PET as well as for the production of activated carbon. Small scale production units for carrying out the pyrolysis, especially modular production units avoid the transport of the biomass and therefore, minimize the cost of handling the waste to central processing units, and allow to minimize the carbon footprint. In addition, the biomass, in particular the food waste, can be processed before degradation occurs. Furthermore, the size of the modular production unit may be chosen dependent on the amount of the lignocellulosic biomass. Preferably, the modular production unit is self-sufficient, that is for example, independent from an external power source.

The energy to operate a production unit, especially a mobile production unit may be obtained at least partly by burning the biochar, the remainder of the crude bio-oil and/or the gases obtained during the pyrolysis process.

In a most preferred embodiment the terephthalic acid obtained by the process according to the present invention may be used for the production of polyethylene terephthalate and for the production of polyaramides. The use for the production of PET is in particular preferred since polyethylene terepthalate is the most important commercial polyester polymer.

### EXAMPLE 1

This illustrative example is the equivalent to six processing stations operating at six different temperatures, wherein the input biomass is fed at 300° C. and the final solid is removed at 600° C.

300 g of lignocellulosic biomass consisting of coconut shells as one specific kind of food waste along with dimethyl ether as co-solvent were devolatilized starting at a temperature of 300° C. and ending at 600° C. with a temperature increment of 60° C. for every hour. 250 g methanol were passed through a silica alumina catalyst to generate the required co-solvent. The aromatic fraction was collected at each temperature for each catalyst at half-hour intervals. A total of 51 ml of aromatic fraction was produced.

The aromatic fraction comprises 32.2% benzene, 37.9% toluene, 12.0% xylene, 8.7% naphtalenes, and 8.7% others. Toluene was separated from the other aromatics by distillation.

### EXAMPLE 2

A mixture containing 0.75 mol of toluene, 0.75 mol of hydrogen fluoride and 0.75 mol of boron trifluoride was cooled in a pressure-resisting vessel at a temperature of −32° C. to 12° C. for about 24 minutes with carbon monoxide at a pressure of 25 bar. A 40.6% conversion to p-tolualdehyde was obtained. The p-tolualdehyde was isolated by distillation after removing the catalyst with several water washes.

### EXAMPLE 3

Used as a reactor was a 400 ml-volume complete mixing type reactor equipped with a stirrer, a peep-hole, a jacket for temperature control, a nozzle for blowing air, an exit for an exhaust gas, an inlet for a stock solution and an exit for withdrawing the product solution.

A stock solution consisting of 200 g of p-tolualdehyde as obtained in Example 2, 10 g of cobalt acetate and 1000 g of glacial acetic acid was charged into the reactor at a rate of 270 ml/hr. The reaction solution was thoroughly mixed by the stirrer and oxygen was blown while the temperature of the inside of the reactor was maintained at 130° C. The reaction was carried out at a reaction pressure of 3 kg/cm2. The vapor phase portion was withdrawn from the reaction system at a rate of 5 l/hour to prevent the accumulation of the gas produced. The product slurry was then withdrawn at such a rate as the quantity of the contents of the reactor might be kept constant.

The obtained terephthalic acid has a purity of 99.0% and was produced in a yield of 95%.

1. Method for producing terephthalic acid from lignocellulosic biomass including the steps of
a. pyrolysis of lignocellulosic biomass in the presence of a catalyst obtaining biochar, crude bio-oil and gases;
b. collecting an aromatic fraction from the crude bio-oil and/or from the gases;
c. extracting toluene from the aromatic fraction by distillation;
d. converting the toluene to a p-tolualdehyde by catalyzed carbonylation;
e. oxidation of the p-tolualdehyde in a liquid phase oxidation to terephthalic acid.

2. Method according to claim 1, wherein the lignocellulosic biomass is food waste, wood residues or municipal paper waste.

3. Method according to claim 1, wherein the lignocellulosic biomass is food waste selected from the group of waste occurring during agricultural production, waste occurring during postharvest handling and storage, waste occurring during processing, waste occurring during distribution and waste occurring during consumption or mixtures thereof.

4. Method according to claim 3, wherein the lignocellulosic biomass is one specific kind of food waste.

5. Method according to claim 1, wherein the lignocellulosic biomass is selected from the group of coconut shells, pecan shells, almond shells, rice straw, wheat straw, rice hulls, corn stover, corn straw, coffee draff, cocoa shells, potato peels and corn crops.

6. Method according to claim 1, wherein the carbon monoxide (CO) is extracted from the gases during the pyrolysis and used for the carbonylation of the toluene to p-tolualdehyde.

7. Method according to claim 1, wherein the pyrolysis is carried out at a temperature of 300° to 500° C., preferably at 325° to 400° C.

8. Method for producing terephthalic acid from lignocellulosic biomass including the steps of
a. pyrolysis of lignocellulosic biomass in the presence of a catalyst obtaining biochar, crude bio-oil and gases;
b. collecting an aromatic fraction from the crude bio-oil and/or from the gases;
c. extracting xylene from the aromatic fraction by distillation;
d. converting the xylene to a mixture of o- xylene, m-xylene, p-xylene and p-tolualdehyde by catalyzed carbonylation;
e. purifying the p-toluadehyde from o-, and m- xylene by freezing at a temperature of −5° C.
f. oxidation of the p-tolualdehyde and the p- xylene in a liquid phase oxidation to terephthalic acid.

9. Method for producing activated carbon by
physical or chemical activation of the biochar obtained in step a) in claim 1, or
physical or chemical activation of the lignocellulosic biomass before carrying out the pyrolysis.

10. Method according to claim 9, wherein the lignocellulosic biomass is coconut shells.

11. Method for producing ethylene glycol by extraction of the carbon monoxide and hydrogen from the gases during the pyrolysis and converting the carbon monoxide and hydrogen to ethylene glycol.

12. Method for producing polyethylene terephthalate by reacting terephthalic acid and ethylene glycol, whereby the terephthalic acid is obtained according to the method of claim 1.

13. Method according to claim 12 by reacting terephthalic acid obtained and with ethylene glycol obtained by extraction of the carbon monoxide and hydrogen from the gases during the pyrolysis and converting the carbon monoxide and hydrogen to ethylene glycol.

14. Use of lignocellulosic biomass for the production of terephthalic acid, for the production of ethylene glycol, for the production of polyethylene terephthalate and/or for the production of polyaramides.

15. Use of lignocellulosic biomass for the production of activated hydrocarbon.

16. Use of lignocellulosic biomass for the production of carbon dioxide, preferably for carbonated beverages.

17. The method according to claim 1, wherein a material selected from the group consisting of biochar, methane, hydrogen and residues of crude bio-oil and mixtures thereof is used as an energy source for the method.

18. A modular production unit configured to carry out the process according to claim 1, comprising at least one pyrolysis unit for the pyrolysis of lignocellulosic biomass.

19. The modular production unit according to claim 18, additionally comprising a cyclone for the collection of biochar.

20. The modular production unit according to claim 19 additionally comprising a kiln to obtain activated carbon.
